(19) United States
(12) Patent Application Publication
(10) Pub. No.: US 2012/0203161 A1
(43) Pub. Date: Aug. 9, 2012

(54) DEUTERATED WATER AND RIBOFLAVIN SOLUTION FOR EXTENDING SINGLET OXYGEN LIFETIMES IN TREATMENT OF OCULAR TISSUE AND METHOD OF USE

(75) Inventor: Satish V. Herekar, Palo Alto, CA (US)

(73) Assignee: SEROS MEDICAL, LLC, Palo Alto, CA (US)

(21) Appl. No.: 13/390,061

(22) PCT Filed: Aug. 12, 2010
(86) PCT No.: PCT/US2010/045356

§ 371 Date: Apr. 24, 2012

Related U.S. Application Data
(60) Provisional application No. 61/233,315, filed on Aug. 12, 2009.

(51) Int. Cl. A61F 9/00; A61P 27/02; A61K 31/525

(52) U.S. Cl. 604/20; 514/251

(57) ABSTRACT

A solution of deuterated water containing a riboflavin-based photosensitizer is provided in order to extend life-times of UVA/Rf photo-generated intra-stromal singlet oxygen, in combination with UVA delivery profiles of pulsing, fractionation, and optionally auxiliary stromal/Rf hyper-oxygenation in order to accelerate protein cross-linking density rates in ocular tissue. A 100% deuterated water solution with 0.1% riboflavin in solution increases singlet oxygen lifetimes by at least an order of magnitude without inducing endothelial cell apoptosis, thereby also permitting use of some combination of lower percentages of deuterated water, lower concentrations of riboflavin or lower dosages of UVA on intact (un-debrided) epithelium for equivalent cross-link densities compared to current acceptable corneal cross-linking procedures. Lower concentrations of deuterated water with regular water, for example, yields shorter singlet oxygen lifetimes in approximately linear proportion to concentration, which are considered acceptable in therapies known or being developed in the art of corneal cross-linking.

This application claims benefit of U.S. Patent Application No. 61/233,315 which was filed on Aug. 12, 2009.

### BACKGROUND OF THE INVENTION

This invention relates to compositions, methods and delivery systems for promoting cross-linking of proteins in tissue using ultraviolet irradiation of a solution of water containing riboflavin or its analogues, particularly in ocular tissue (such as tissue in the sclera, cornea, prepapillary region, etc.).

Therapies are known or are under laboratory investigation for promoting structural enhancement of stromal and scleral tissues by application of ultraviolet A radiation to riboflavin in a water solution on ocular tissue in the presence of oxygen-containing atmosphere. The present inventor has determined that singlet oxygen lifetimes have an evident impact on degree of cross-linking densities of protein such as collagen, a main structural component of stromal and scleral tissues.

Literature reports that deuterated water can increase singlet oxygen lifetimes in various methods for generating singlet oxygen. This invention takes advantage of this discovery in a new context. A search of the literature has found no reports or suggestions of the present methodology and compositions. Reference is made to the collection of references supplied by the inventor to the Patent and Trademark Office for consideration.

Collagen cross-linking (CXL) in ophthalmology, as it currently exists in Europe (where it is approved), provides a biomechanical basis of increased corneal strength (i.e., stability & stiffness) as a result of the formation of covalent bonding between collagen strands. This occurs when a photo-sensitizer, riboflavin (Vitamin B-2) is applied to the de-epithelialized surface of the cornea. This epithelial protective tissue over the cornea is surgically debrided (i.e., surgically removed) so the riboflavin can pass (i.e., be absorbed) into the stroma (collagen layers) of the cornea. After the riboflavin saturates the stroma, it is exposed to UVA light (approximately 365 nm). This excitation of the riboflavin by the UVA results in the creation of free radicals that interact with amino acids and carbonyl groups in neighboring collagen molecules to form the strong covalent chemical bonds. Debride refers to removal of dead, contaminated or adherent tissue or foreign material.

The primary emphasis in the application of CXL for ophthalmology has been in the treatment of keratoconus, which is prevalent in about one in 2,000 people in the US and Europe, with a slightly higher percentage in Asian countries. This condition is manifested by a weak cornea which becomes too elastic and stretches, causing it to bulge forward. This changes the curvature of the cornea which almost always leads to poor visual acuity (not correctable with glasses and/or soft contact lenses) that requires the use of rigid gas permeable lens. Thus, when the cornea begins losing its shape (i.e., becomes cone shaped instead of spherical) the person typically becomes nearsighted and will develop irregular astigmatism, which causes the blurring of vision. As this condition progresses, this person may develop scarring and a very irregular corneal curvature. If the person cannot be helped with the rigid contact lens, then he/she will require a corneal transplantation.

There are other conditions/corneal diseases where the cornea can become stretched and distorted. One of these, where CXL is currently being utilized, is in corneal ectasia. This condition involves stretching of the cornea (collagen tissue) that occurs after refractive surgeries, such as laser in situ keratomileusis (LASIK) or photorefractive keratectomy (PRK). Other corneal diseases in which CXL has been tried successfully include corneal ulceration (possible sequelae to bacterial, viral or fungal infections) and bullous keratopathy (excess fluid accumulation causing corneal edema).

The existing procedure of CXL has been clinically proven (in Europe) to be safe. However, in its current form, the procedure is very rudimentary with a number of significant limitations including but not limited to: the procedure takes too long (approximately one hour in total); removal of the corneal epithelium (i.e., debridement) is required, making the procedure invasive and uncomfortable for the patient intra-operatively and for 3-4 days following surgery; and, it is not fully measurable for accuracy. These limitations clearly preclude the use of CXL for many corneal treatments that would require a fast and highly accurate process for stiffening and stabilizing the cornea.

### SUMMARY OF THE INVENTION

According to the invention, a solution of deuterated water containing a riboflavin-based photosensitizer (Rf aka Vitamin B2) is provided in order to extend lifetimes of UVA/Rf photo-generated intra-stromal singlet oxygen, in combination with UVA delivery profiles of pulsing, fractionation, and optionally auxiliary stromal/Rf hyper-oxygenation in order to accelerate protein cross-linking density rates in ocular tissue.

This invention is based upon the discovery that there is a correlation between the concentration of dissolved [singlet] oxygen in irradiated ocular tissue and the efficiency of cross-linking with the photo-sensitizer riboflavin. Our studies have demonstrated that a 100% deuterated water (D2O) solution with 0.1% riboflavin in solution increases singlet oxygen lifetimes by about an order of magnitude (10× or more). Further studies have shown that the such application of deuterated water does not induce endothelial cell apoptosis.

Our studies have also shown that by delivering optimized combinations of deuterated water, riboflavin, and UVA dosage on an intact (undebrided) epithelium, equivalent cross-link densities are rapidly achieved with reduced adverse effects as compared to current treatments. Lower concentrations of deuterated water with regular water, for example, yields shorter singlet oxygen lifetimes. These lifetimes have approximately a linear relationship to the concentration of deuterated water. These lower concentrations are considered acceptable in therapies known or being developed in the art of corneal cross-linking.

Our experiments have shown various correlations such as between the following: the concentration of D2O and reactive oxygen species (ROS) lifetimes; the UVA fluence and ROS concentration; and, the dissolved oxygen consumption and UVA fluence.

Deuterated water refers to water containing a higher-than-normal proportion of the hydrogen isotope deuterium, either as deuterium oxide, D2O or ²H2O, or as deuterium protium oxide, HDO or ¹H²HO. Conventional water is water that has a normal proportion of deuterium isotope, such as in tap water to distilled water.

The invention will be better understood by reference to the following detailed description in connection with the accompanying drawings.

### BRIEF DESCRIPTION OF THE DRAWINGS

FIGS. 1A-1C are illustrations of a first method according to the invention.

FIGS. 2A-2D are illustrations of a second method according to the invention.

FIG. 3 is a schematic diagram of a delivery system according to the invention.

FIG. 4 is a graph showing the relationship between D2O and ROS lifetimes.

FIG. 5 is a graph showing the relationship between ROS concentration and UVA irradiation (fluence).

FIG. 6 is a graph showing the relationship between rate of oxygen consumption and UVA irradiation.

### DETAILED DESCRIPTION OF THE INVENTION

The invention is embodied in methods, compositions and delivery systems, particularly in relation to therapies for strengthening and re-shaping ocular tissue. The formulation invention includes a composition or substance comprising a solution of deuterated water (between 100 wt % D₂O and 10 wt % D₂O in water) containing a riboflavin based photo-sensitizer, carboximethylcellulose (CMC), and benzalkonium chloride (BAC).

In one embodiment, the riboflavin based photo-sensitizer is Rf aka Vitamin B2. It should be noted that all concentrations are, unless otherwise specified, wt/vol (for example, 0.1% Rf refers to ˜0.1 gm in 100 mL). The molecular weight of Rf is: ˜378 gm/L. In some embodiments, the concentration of the riboflavin based photo-sensitizer, such as Rf aka Vitamin B2, is between X and Y, or more narrowly, V and W. In some embodiments, the concentration of carboximethylcellulose, is 0.2% or about 0.2%. In some embodiments, the concentration of carboximethylcellulose is between X and Y, or more narrowly, V and W. In some embodiments, the concentration of BAC, is 0.2% or about 0.2%. In some embodiments, the concentration of BAC is between X and Y, or more narrowly, V and W. In certain embodiments, the formulation invention includes deuterated water between 100% D₂O and 10% D₂O in water containing, the riboflavin based photo-sensitizer Rf aka Vitamin B2 of about 0.1% Molar concentration, carboximethylcellulose (CMC) of about 0.2%, and benzalkonium chloride (BAC) of about 0.02%. Embodiments of the invention include any value or range of D2O between 10% and 100% in the formulation.

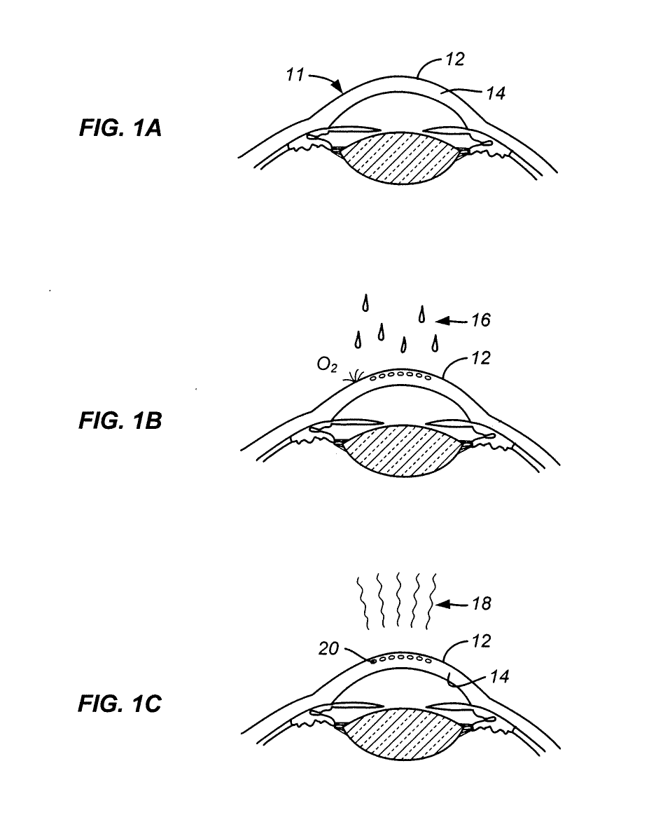

Referring to FIGS. 1A-1C, a method according to the invention is illustrated. FIG. 1A depicts an intact cornea 11 comprising an epithelium 12 with underlying stromal tissue 14. As shown in FIG. 1B, the formulation 16 according to the invention is applied as a spray or droplets to the epithelium 12 in the presence of ambient oxygen (in the air) to an undebrided corneal surface. The period of exposure of formulation is several minutes. In some embodiments, the period of exposure can be between 1 to 2, 2 to 3, 3 to 5, 5 to 7, 7 to 10, or greater than 10 minutes. Bursts of spray or droplets are applied over the affected area for the duration of the soaking cycle.

Then the solution-soaked stromal region 14 is irradiated with ultraviolet A 18, as shown in FIG. 1C. The UVA irradiation treatment may be continuous (i.e., irradiation without interruption) for a period ranging from 1 to 15 minutes or fractionated (turned on and off for a few seconds to a minute) or pulsed (brief bursts of high irradiance with ON times in the 1 microsecond to millisecond range, and frequencies in the 1 killohertz to 500 killohertz range. The irradiation creates reactive oxygen species (ROS) that cause the desired crosslinking of proteins 20. In one preferred embodiment, which maximizes benefits efficiently, the irradiation is pulsed and fractionated, to promote the production of singlet oxygen, or reactive oxygen species (ROS) in the intrastromal region to thereby promote the desired cross-linking of proteins 20 during the lifetimes of the reactive oxygen.

In embodiments of the invention, there may be various compounds which can function as both preservative and penetration enhancers. These compounds include, but are not limited to benzalkonium chloride (BAC) and sodium ethylenediaminetetraacetate (EDTA), as, well as viscosity agents such as carboxymethylcellulose (CMC) or dextran. BAC (˜0.02%) and EDTA (˜0.1%) enhance penetration of the riboflavin and D₂O water formulation. CMC (˜0.2%) or dextran (˜20%) enhance the lubricity and the formation of a persistent, broader, and more uniform corneal tear film before and during the procedure. This allows greater absorption of the active ingredients of the formulation into the cornea.

Significantly, the riboflavin formulation can also be manufactured with a high concentration of dissolved oxygen. This oxygen enrichment enables the production of greater ROS concentration in a shorter period of time, and, in turn, this makes higher UVA irradiance practical. However, it should be noted that there may be other means to diffuse oxygen gas into the stroma, which might include, among others, the use of a device that would deliver such oxygen gas to the corneal surface. This oxygen gas then diffuses (albeit slowly) into the stroma, thereby increasing dissolved oxygen. In summary, the ability to increase dissolved oxygen in the stroma enables the use of a higher UVA irradiance exposure to the collagen tissue. This concept of embedding dissolved oxygen in the stroma means that optimum cross-linking (i.e., adequate stiffness of the cornea with minimal side effects) can be achieved in a shorter period of time. A means to manufacture the enriched oxygen deuterated Rf formulation, that will provide up to and over 1 year of extended shelf life, is contemplated by this invention. The components of the formulation invention, which are set forth hereinabove, may be optimized for penetration rate, pH, hypotonicity, and lubricity by the proportions that each component is used within the formulation.

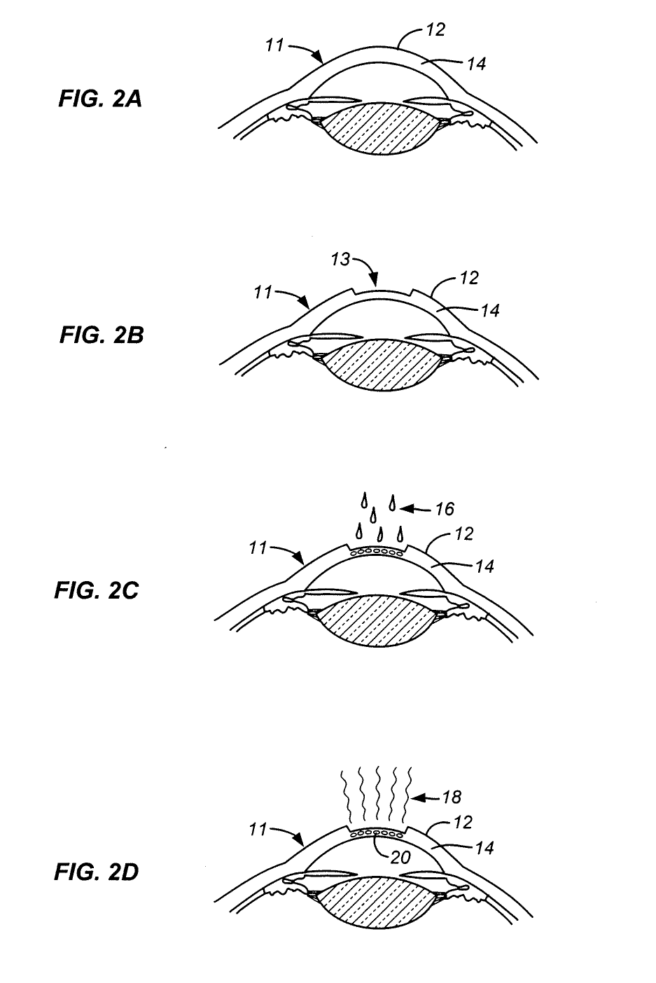

Referring to FIGS. 2A-2D, a further method according to the invention is illustrated. The intact cornea 11 (FIG. 1A) comprises an epithelium 12 with underlying stromal tissue 14. The corneal surface is debrided to remove the surface layer and expose the underlying tissue (FIG. 2B) in a debrided region 13. A solution 16 according to the invention is applied as a spray or droplets to the debrided region 13 in the presence of ambient oxygen (in the air) to the (FIG. 1B). The period of exposure is several minutes. The same ranges of exposure disclosed in the embodiments of FIGS. 1A-C apply to FIGS. 2A-D. Bursts of spray or droplets are applied over the affected area for the duration of the soaking cycle. Due to the debriding, the stromal tissue 14 is soaked to a greater depth than the embodiments of FIGS. 1A-C. Then the solution-soaked stromal region 14 is irradiated with ultraviolet A 18 (FIG. 2D). As described above, the UVA irradiation treatment may be continuous or fractionated (turned on and off for extended periods) or pulsed (brief bursts of high illumination for an extended period), or most preferably pulsed and fractionated, to promote the production of singlet oxygen, or reactive oxygen species (ROS) in the deep intrastromal region to thereby promote the desired cross-linking of proteins 20 during the lifetimes of the reactive oxygen.

The process of soaking the formulation, on either a debrided or undebrided surface, results in diffusing oxygen into the stroma. For a undebrided surface, the penetration is to a depth of up to about 0.5 mm. The penetration into debrided surfaces is greater than 0.5 mm. The UVA irradiation in the presence of oxygen promotes singlet oxygen species generation. The deuterated water with riboflavin extends lifetimes of UVA/Rf photo-generated intra-stromal singlet oxygen This in combination with UVA delivery profiles of pulsing, fractionation, and optionally auxiliary stromal/Rf hyper-oxygenation accelerates protein cross-linking density rates in the ocular tissue. Our studies have shown that the use of a 100% deuterated water solution with 0.1% riboflavin in solution increases singlet oxygen lifetimes by at least an order of magnitude (10× or more) without inducing endothelial cell apoptosis. It is well known in the arts that the current cross-linking procedure may induce the following side effects: (1) stromal haze due to keratocyte apoptosis; (2) endothelial cell density loss.

In another embodiment, the formulation includes a combination of lower percentages of deuterated water, lower concentrations of riboflavin or lower dosages of UVA on intact (un-debrided) epithelium may be employed for equivalent cross-link densities as compared to current acceptable corneal cross-linking standards for CXL procedures. In various embodiments, the ranges of components and delivery parameters of the formulation are as follows: 100% D2O to 1%; 0.1% Rf to 0.01%; 0.02% BAC to 0.01%; 0.2% CMC to 0.1%; 5.4 J/cm2 UVA to 2.5J/cm2; 30 minutes or less UVA exposure.

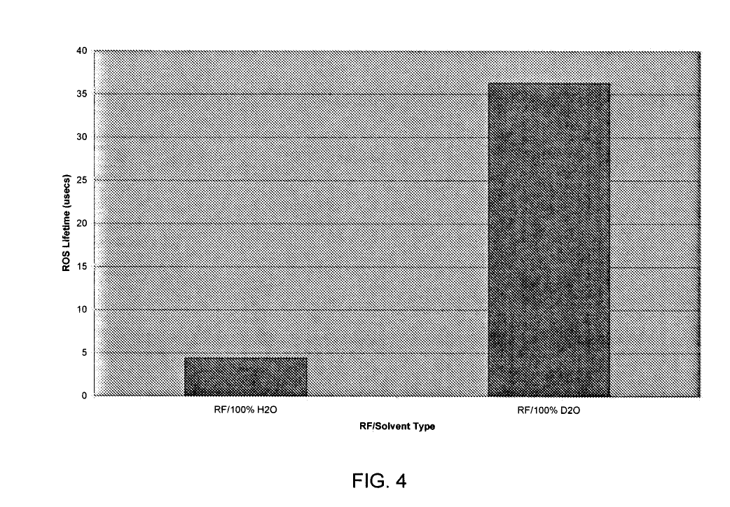

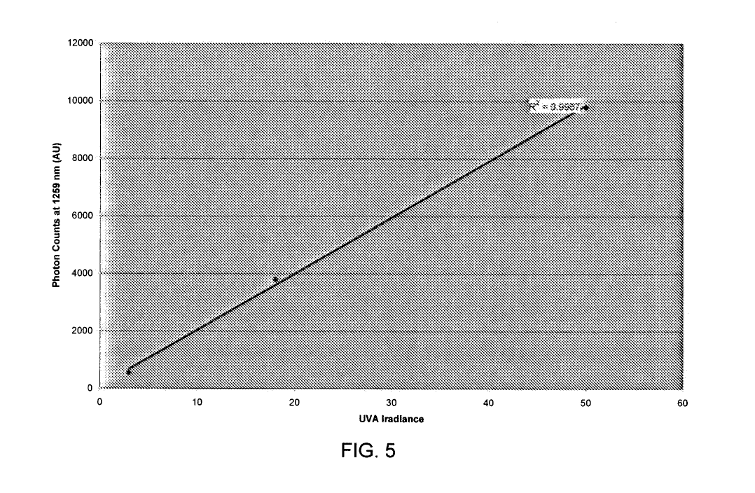

FIG. 4 shows the singlet oxygen lifetime in deuterated water as a function of concentration of D2O. FIG. 4 demonstrates the correlation of ROS lifetimes to varying D2O solvent (0% to 100%) in the 0.1% Rf solution under normoxic (i.e., ambient oxygen dissolved into the test sample at room temperature by natural diffusion conditions in collagen and 0.1% Rf matrices. As shown in FIG. 4, lower concentrations of deuterated water with regular water, for example, yield shorter singlet oxygen lifetimes. The relationship between singlet oxygen lifetime and D2O concentration in regular water is approximately linear, as shown in FIG. 5. This data was generated by a custom built photon counter and dissolved oxygen probe, which was excited by a frequency tripled Nd:Yag laser for time-resolved measurements.

The inventor has measured reactive oxygen species (ROS) in vitro in aerated collagen and riboflavin under UVA illumination and has found an increase in the ROS duration of about 4.5 μSecs for H₂O with no D2O to a duration of over 45 μSecs for a 100% deuterated solvent D₂O (See FIG. 5). FIG. 5 shows a strong linear correlation of ROS concentration in a normoxic collagen and Rf matrix as UVA irradiance is varied from about 3 mW/cm² to about 50 mW/cm².

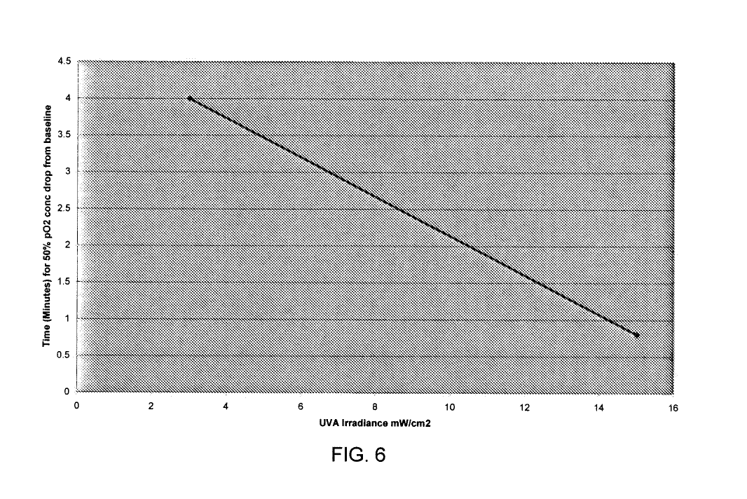

FIG. 6 shows the inverse correlation of dissolved oxygen concentration (due to consumption from varying ROS generation) with varying UVA irradiance in a normoxic collagen+0.1% Rf matrix. A 500% factor is shown in the example below.

A system using dual UVA/Blue sources is able to provide pulsed irradiances up to 150 mW/cm², with pulsing frequencies at up to 200 kHz and is, for example, set to deliver pulses at a 20 kHz (50 μSecs) pulse repetition frequency, and a duty cycle of about 20% (intermittency). This is a 40 μSec UVA OFF period and a 10 μSec high intensity UVA ON period applied cyclically. It is believed that the 10 μSec UVA ON pulse rapidly generates a maximal new population of ROS molecules in the targeted stroma just as the previously stimulated ROS population is about to be depleted or otherwise be consumed through quenching mechanisms in the local microenvironment. It is believed that the 40 μSec UVA OFF period provides sufficient time for chemical interactions in the microenvironment to effect cross-linking of proteins, specifically collagen, in the target region. The singlet oxygen population in the presence of the aerated deuterated solvent survives for an extended duration of about 40 μSecs, while the UVA is OFF.

In a specific embodiment, during the extended reactive lifetime of singlet oxygen, rapid cross-linking reactions are induced in the carbonyl (aldehyde) groups of collagen while dissolved oxygen O₂, riboflavin, and singlet oxygen species (ROS) are consumed (as long as present in sufficient concentrations) by Type II (energy transfer) mechanisms. (From photo-chemistry competitive mechanisms of radicals formation are known: electron transfer or Type I; and, energy transfer, Type II.) This on-off cycle repeats every 50 uSecs (at 20 kHz). Analogues of riboflavin may also be employed, such as 3-methyl-riboflavin tetraacetate.

As riboflavin molecules degrade and transform through such singlet oxygen regenerative timing cycles, they generate reduced fluorescence intensity in the 530 nm-570 nm band in response to UVA which may thereby signal a riboflavin “reinstillation” cycle.

In addition to increased endothelial safety due to reduced riboflavin concentration requirements, the use of viscous carboxy-methyl-cellulose (CMC) in the present formulation forms a corneal film of thickness ˜50 μM-200 μM, which provides added UVA protection to the endothelium. Pulsed UVA applied as herein described (instead of CW UVA) provides for a reduced apoptotic effect on both keratocytes and endothelial cells.

The formulation (D₂O+CMC+BAC) provides for faster penetration and clearance, reducing pre-treat soak times and end-product clearance periods. The use of BAC as a penetration enhancer has been previously reported in the literature.

The rate of diffusion of dissolved oxygen through the stroma depend on corneal thickness, epithelialization state (whether or not debrided), sensitizer pre-oxygenation, viscosity and ambient oxygen environment of the stroma. Some amount of dissolved oxygen will continue to migrate into the stroma and sclera. However, during UVA irradiation a much larger consumption of local dissolved oxygen occurs than can be supplied through ambient diffusion The formulation, and the use of UVA pulsation and fractionation is able to overcome the dissolved oxygen limitations inherent in ambient diffusion. Depending on the depth of cross-linking desired, a pause in the UVA irradiation (of the order of seconds to minutes) cycle may permit dissolved oxygen to permeate deeper in the stroma before localized consumption due to ROS generation.

Total cross-linking treatment times and singlet oxygen/riboflavin molecular efficiencies are significantly enhanced due to this timed UVA/oxygen modulation sequencing with minimized UVA dosage but with minimal or no loss in effectivity and little or no increase in toxicity.

Singlet oxygen concentration as generated according to this method is highly linearly correlated to UVA irradiance. FIG. 6 shows the rate of dissolved oxygen consumption in the collagen at 15 mW/cm² in two test samples. Each test sample included collagen and 0.1% Rf solution. FIG. 6 shows the inverse correlation of dissolved oxygen concentration (due to consumption from varying ROS generation) with varying UVA irradiance in a normoxic collagen+0.1% Rf matrix. A 500% modulation factor is shown in the graph below. Here we show that a 500% increase in UVA irradiance increases the rate of dissolved oxygen consumption by 500%. Increased UVA (See FIG. 6) irradiance also generates correspondingly increased cross-link densities with correspondingly greater dissolved oxygen consumption during exposure.

While the main focus persistent in prior art publications is on collagen cross-linking in the stroma, the inventor has concluded that extra-cellular matrix (ECM)/proteoglycans may play a role in the stromal cross-linking process and may form inter-molecular and intra-molecular collagen/proteoglycan cross-links. The object of this proposed method includes such cross-linking as well.

D₂O is non-toxic and is readily available. One supplier is Sigma Aldrich, from which a 10 gram vial costs about $40.

A generalized formulation for cross-linking according to the invention may be characterized as: a) an effective amount of a penetration enhancing agent; b) an effective amount of a viscosity agent which maintains film thickness and extends UV protection c) an effective amount of an agent imparting a hypotonic solution (i.e., a solution which has an osmolarity less than ˜295 mOsol, and is adjusted by the salt NaCl); d) an effective amount of an agent for extending singlet oxygen lifetimes, e) an effective amount of a photosensitizing agent, and, f) an effective amount of deuterated water forming a solution. The formulation is configured upon delivery to ocular tissue (through its delivery mechanism and the like) for reaction with UVA irradiation directed (via a lamp or fiber) at the ocular tissue in the presence of oxygen (such as ambient air). The lifetimes of singlet oxygen released by the UVA radiation for promoting protein cross-linking in the ocular tissue are extended by the formulation. The viscosity agent imparting viscosity control may be or contain CMC at a concentration between [1%] and [90%]. The penetrating enhancing agent may be or contain 0.02% or less BAC, and the photosensitizing agent may be or contain riboflavin or its analogues.

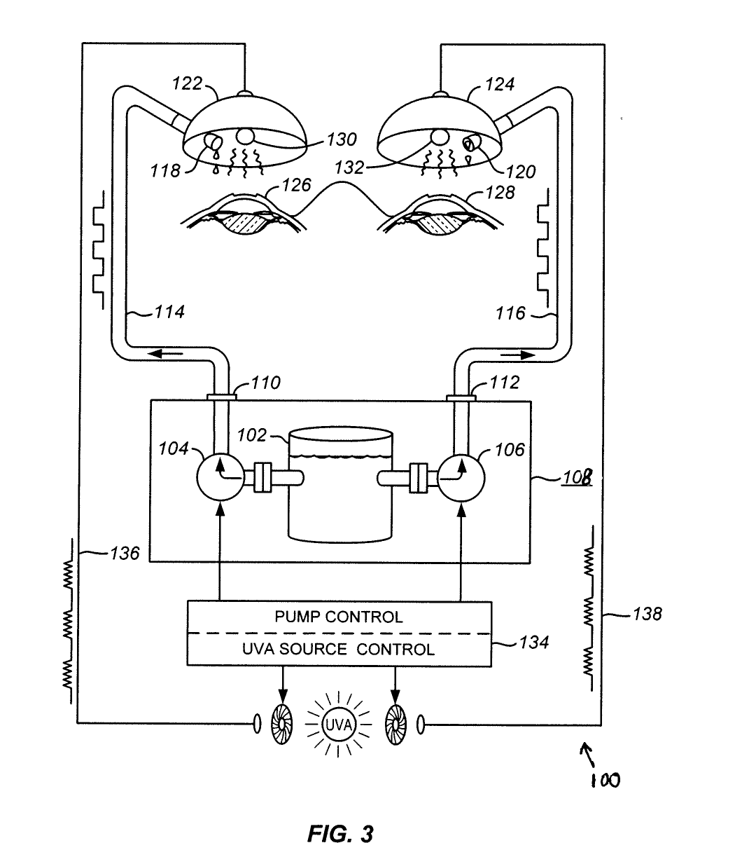

Referring to FIG. 3, an appropriate delivery system 100 may be the content of a substance in a single use dose container 102 and an appropriate applicator subsystem comprising one or more medical grade peristaltic pumps 104, 106 in a housing 108 having outlets 110, 112, coupled via tubes 114, 116 to a pair of spray dispensing devices 118, 120 each to be mounted on frame 122, 124 over an eye 126, 128 of a patient to provide sterile delivery of the substance to the affected area of each eye, a region about 8 mm in diameter. Irradiation ports 130, 132 mounted to the frame 122, 124 provide directed radiation, which is controlled by a UVA source and controller 134 that delivers the prescribed irradiation dosage (e.g., fractionated pulsed UVA for a period of a few minutes) via fiber optic cables 136, 138. The same controller 134 may be coupled to and control the pumps 104, 106 to meter the delivery of the solution according to the invention. The delivery system provides for dual delivery of the formulation, i.e., delivery simultaneously to both eyes. The system further provides for dual irradiation of UVA to each eye simultaneously. Although delivery and irradiation to one eye or sequentially is also an embodiment of this invention.

Features of the invention are advantageous when exciting the sensitizer, since one can select the duty cycle with an OFF time of about 50 μsecs (ROS lifetime). The peak amplitudes can be dynamically set from 3 mW/cm² to >100 mW/cm² and at up to 100 kHz frequency. A simple nomogram might be: 10 kHz Pulsing Frequency with 50% duty cycle (50 μsecs ON/50 μsecs OFF).

This invention has been explained with respect to specific embodiments. Other embodiments will be evident to those of ordinary skill in the art. For example, cross-linking may be employed for treatment of maladies or used in procedures including keratoconus, myopia, presbyopia, LASIK, cataract, and corneal transplantation. Therefore, it is not intended that the invention be limited, except as indicated by the amended claims.

What is claimed is:

1. A substance for ocular treatment comprising:
carboxy-methyl-cellulose for providing viscosity control and protection against incident ultraviolet radiation;
an effective amount of benzalkonium chloride (BAC) as a penetration enhancer of the substance into ocular tissue;
an effective amount of deuterated water for extending singlet oxygen lifetimes; and
an effective amount of a riboflavin-based photosensitizer in solution with the deuterated water and carboxy-methyl-cellulose,
said solution being configured for reaction with ultraviolet A radiation directed at ocular tissue in the presence of oxygen, such that the lifetimes of singlet oxygen released by the ultraviolet A radiation are extended for promoting protein cross-linking in the ocular tissue.

2. The substance according to claim 1 further including conventional water in mixture with the deuterated water, the deuterated water exceeding one percent of the total solution.

3. The substance according to claim 1 wherein the concentration of D2O in the solution is between 10% and 100%.

4. A substance for ocular treatment comprising:
a) an effective amount of a viscosity agent for film thickness control and UV protection;
b) an effective amount of an agent imparting a hypotonic solution; [what does hypotonic solution mean]
c) an effective amount of an agent for extending singlet oxygen lifetimes;
d) an effective amount of a photosensitizing agent that aborbs UV radiation;
e) an effective amount of deuterated water forming a solution;
said solution being configured for reaction with ultraviolet A radiation directed at ocular tissue in the presence of oxygen, such that the lifetimes of singlet oxygen released by the ultraviolet A radiation are extended for promoting protein cross-linking in the ocular tissue.

5. The substance according to claim 4 wherein the viscosity agent imparting viscosity control comprise CMC at a concentration between [1%] and [90%].

6. The substance according to claim 4 wherein the photosensitizing agent comprises riboflavin.

7. The substance according to claim 4 further including conventional water in mixture with the deuterated water, the deuterated water exceeding one percent of the total solution.

8. A delivery system comprising:
the substance of claim 4;
a single use dose container containing the substance; and
an applicator for sterile delivery of the substance,
wherein the applicator comprises tubes fluidly connecting the container to a pair of spray dispensing devices,
wherein each pair of spray dispensing devices is mounted on a frame,
wherein each of the frames is configured to be disposed over an eye of a patient to provide sterile delivery of the substance to an affected area of each eye, and
wherein irradiation ports are mounted to each of the frames to provide directed radiation controlled by a UVA source.

9. A method for promoting cross-linking of proteins in ocular tissue comprising:
applying a solution comprising an effective amount of deuterated water and an effective amount of riboflavin as a photosensitizer in solution with the deuterated water to ocular tissue in the present of oxygen; and
irradiating the ocular tissue with ultraviolet A radiation to effect creation of singlet oxygen for reaction with protein forming the ocular tissue in order to effect protein cross-linking in the ocular tissue.

10. The method according to claim 9, wherein the protein being collagen.

11. The method according to claim 10, wherein the solution being externally applied to the ocular tissue.

12. The method according to claim 9, wherein the irradiation being pulsed.

13. The method according to claim 12, wherein the pulsed irradiation being intermittent.

14. The method according to claim 9, further including a preparatory step, the preparatory step comprising debriding the ocular tissue in a treatment region to promote deeper infiltration of stromal tissue by the solution.

15. The method according to claim 9, wherein the solution further comprises carboxy-methyl-cellulose (CMC), wherein the CMC provides viscosity control and protection against damage to the ocular issue from the ultraviolet radiation radiation.

16. The method according to claim 9, wherein the solution further comprises benzalkonium chloride (BAC) which enhances penetration of the solution into ocular tissue.
